(19) United States
(12) Patent Application Publication
(10) Pub. No.: US 2026/0207949 A1
(43) Pub. Date: Jul. 23, 2026

(54) CERTIFICATE-BASED ELLIPTIC CURVE DIFFIE-HELLMAN AUTHENTICATION USING PUBLIC KEY INFRASTRUCTURE FOR IMPLANTABLE DEVICES

(71) Applicant: Greatbatch Ltd., Clarence, NY (US)

(72) Inventors: Federico Nin, Montevideo (UY); Andres Duarte, Montevideo (UY); Oscar Sanz, Montevideo (UY); Felipe A. Saravia, Montevideo (UY)

(21) Appl. No.: 19/448,874

(22) Filed: Jan. 14, 2026

Related U.S. Application Data
(60) Provisional application No. 63/746,298, filed on Jan. 17, 2025.

(51) Int. Cl. A61N 1/372

(52) U.S. Cl. CPC A61N 1/37254; A61N 1/37276

(57) ABSTRACT

A method of mutual authentication between an implantable medical device (IMD) and an external device (ED) includes putting the IMD and the ED into a numeric comparison authentication mode; performing secure pairing that includes the IMD and ED exchanging public keys; producing, by both the IMD and ED, a respective confirmation value using both the public keys, and both the IMD and ED signing its respective confirmation value using its own private signing key; exchanging signed confirmation values; and continuing communication between the IMD and ED when both the IMD and ED verify a received signed confirmation value and ending communication when at least one of the IMD and ED does not verify the received signed confirmation value.

### CLAIM OF PRIORITY

This application claims the benefit of U.S. Provisional Application No. 63/746,298 filed on Jan. 17, 2025, which is hereby incorporated by reference in its entirety.

### TECHNICAL FIELD

Embodiments of the disclosure relate generally to body-implantable devices and more specifically to secure communication between a body-implantable medical device and an external device.

### BACKGROUND

Implantable medical devices include but are not limited to implantable pacemakers, implantable cardioverter defibrillators, implantable neurostimulators, and implantable heart pumps. The devices can be used to treat patients or subjects using electrical stimulation therapy or other therapy, and to aid a physician or caregiver in patient diagnosis through internal monitoring of a patient's condition. Some implantable medical devices can be diagnostic-only devices, such as implantable cardiac monitors, implantable loop recorders, and implantable heart failure monitors. Patient status can be monitored by uploading diagnostic information from the implantable device to an external device. Patient treatment can be adjusted by programming changes in parameters related to detection of the patient's condition and the therapy provided by the implantable device. Communication between an external device and an implantable device should be secure to prevent unauthorized access to the implantable device.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, various embodiments discussed in the present document.

FIG. 1 is a block diagram of portions of an example of a medical device system.

FIG. 2 is a block diagram of portions of an example of an implantable medical device and an external device.

FIG. 3 is a flow diagram of an example of a method of communication between an implantable device and an external device.

FIGS. 4A-4B show an example of communication between an implantable medical device and an external device.

### DETAILED DESCRIPTION

Embodiments of the present disclosure are directed to radio frequency (RF) communication with implanted medical devices (IMDs). Some IMDs communicate with external equipment using middle-range (e.g., within a few meters) RF transceivers. Such middle-range communication may be susceptible to unauthorized communication (e.g., man-in-the-middle attacks). Unauthorized individuals could potentially gain access to the IMD, enabling them to send dangerous commands or intercept communications. Such breaches pose severe risks to both patient privacy and health, as attackers could manipulate device settings or access sensitive patient medical data.

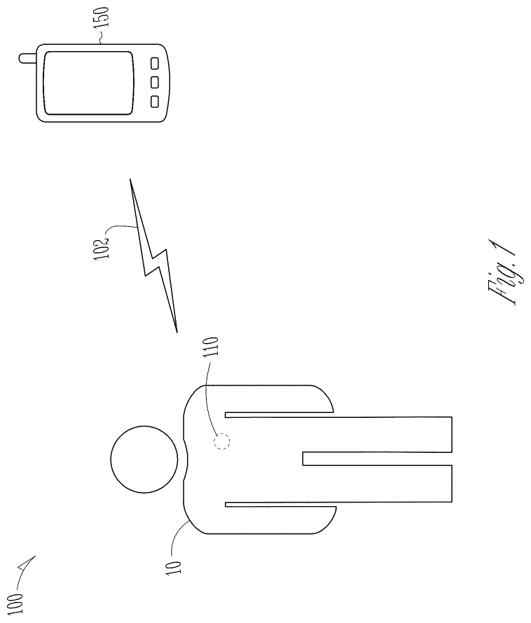

FIG. 1 is a block diagram of portions of an example of a medical device system 100 that includes an IMD 110 and an external device or ED 150. The IMD 110 and ED 150 communicate wirelessly using RF communication 102. The IMD 110 may be a diagnostic-only device that accumulates physiological information about the patient 10 using sensors of the IMD 110. Physiological information can be uploaded by the ED 150 for analysis or to relay to a separate device for analysis. The IMD 110 may be a therapeutic device. For instance, the IMD 110 may include stimulation circuitry that provides electrical stimulation therapy to a patient 10 when connected to electrodes. The IMD 110 may provide pacing stimulation therapy, cardioversion therapy, defibrillation therapy, or neurostimulation therapy. The IMD 110 may sense physiological electrical signals associated with the heart, spine or brain of the patient 10 and deliver electrical stimulation therapy based on the sensed signals. The IMD 110 includes communication circuitry to communicate information with the ED 150. The ED 150 may be a smartphone or a computer. The ED 150 may be a programming device that communicates one or more wireless signals with the IMD 110, such as by using radio frequency (RF) or by one or more other telemetry methods. The ED 150 can communicate information with the IMD 110 to configure operation of the IMD 110 by downloading operating parameters to the IMD 110, and to upload data recorded by the IMD 110 without removal of the IMD 110.

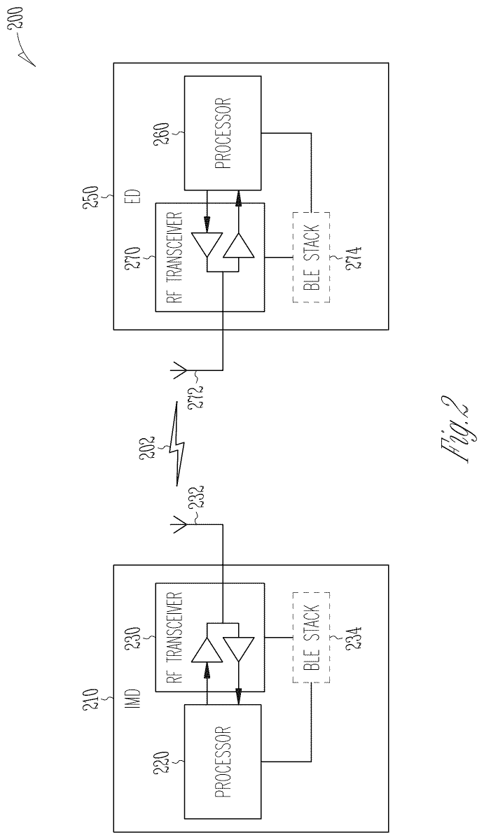

FIG. 2 is a block diagram of portions of an example of an IMD 210 and an ED 250. The IMD 210 may be a pacemaker, cardioverter-defibrillator, neurostimulator, heart pump, or other implantable device. In some examples, the IMD 210 is a diagnostic-only device that records sensed physiological signals using sensors of the IMD 210. The IMD 210 includes processing circuitry such as one or more processors 220 or microcontrollers to control the delivery of therapy and recording of physiological information. The processor 220 can include one or more of a microprocessor, an application specific integrated circuit (ASIC) programmable gate array (PGA), or other type of processor that executes instructions included in software or firmware contained in memory of the IMD 210.

The processor 220 is operatively coupled to an RF transceiver 230 and an RF antenna 232 to communicate information to the ED 250 using RF communication 202. The RF transceiver 230 may be included in a Bluetooth Low Energy (BLE) system on chip (SoC) and the IMD 210 may include a multilayer BLE stack 234 that can include an application host layer and controller layer. The individual layers of the BLE stack 234 may be implemented by the processor 220 or the BLE system. The processor 220 of the IMD 210 communicates information with the ED 250 using the RF transceiver 230. The ED 250 includes processing circuitry such as one or more processors 260. The ED 250 includes an RF transceiver 270 and RF antenna 272 to communicate information to the IMD 210. The ED may also include a BLE stack 274, and the IMD 210 and ED 250 may communicate using a BLE protocol.

The communication distance between the devices may be middle range or longer range, and the communication does not rely on close proximity (as with near field communication for example) as evidence of authorized access. The medical device system 200 implements a public key infrastructure (PKI) framework for mutual authentication between devices. If the IMD 210 and ED 250 communicate using the BLE protocol, at least a portion of the steps of the PKI framework may be integrated within the BLE stacks of the IMD 210 and ED 250.

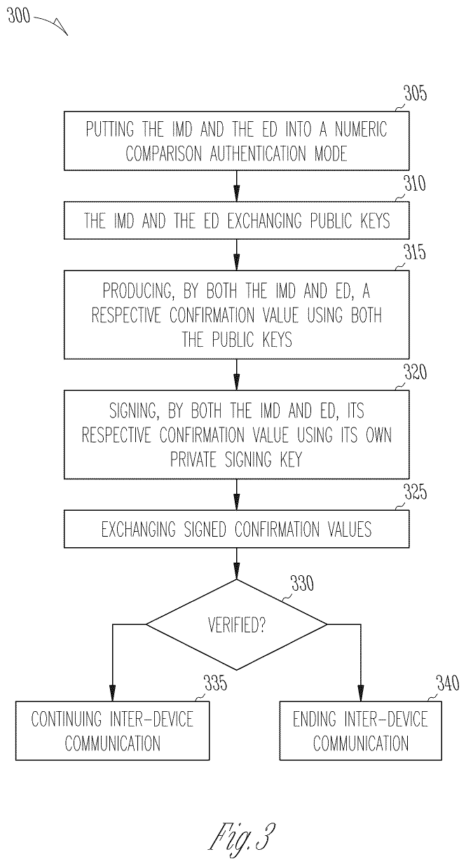

FIG. 3 is a flow diagram of an example of a method 300 of mutual device authentication between the IMD 210 and the ED 250 of the medical device system 200. The system may also include a local server or cloud server (not shown) functioning as a Certification Authority (CA). Both the IMD and ED begin with assigned private/public key pairs, certificates (public keys signed by the CA), and the CA public key. Each of the IMD 210 and ED 250 possesses its own digital certificate for identity authentication. Numeric comparison is used for device authentication.

At block 305, the IMD 210 and the ED 250 are put into a numeric comparison authentication mode in which the devices exchange their pairing features. Both of the devices set specific Input-Output (IO) capabilities even though one or both of the IMD 210 and the ED 250 do not have the IO capabilities. This causes the processing circuitry of the devices to enter the numeric comparison mode as the authentication method.

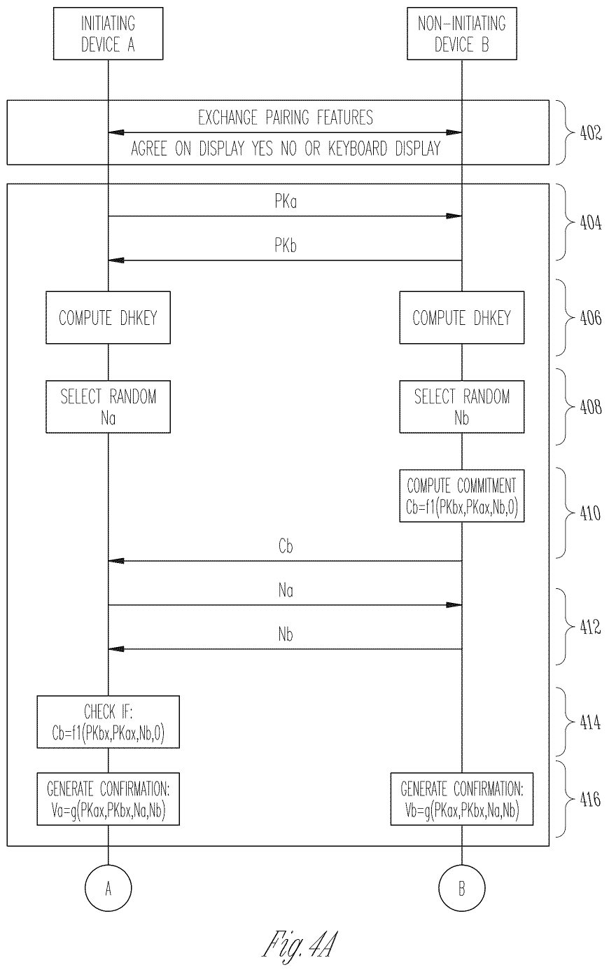

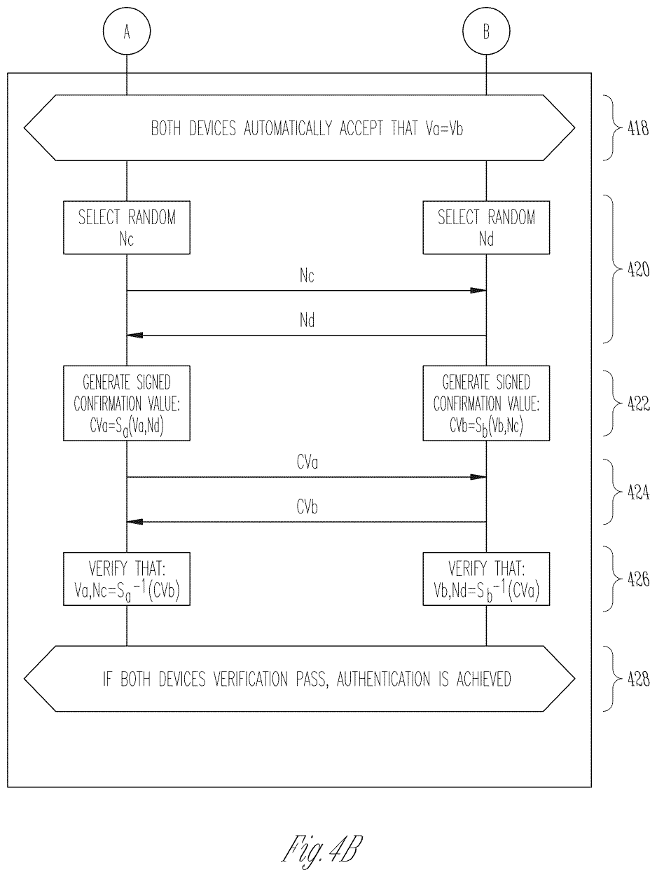

FIGS. 4A-4B is a protocol sequence diagram of an example of communication flow between the IMD 210 and the ED 250. The diagram shows an exchange of communication between an Initiating Device A and a Non-Initiating Device B. Either of the IMD 210 or the ED 250 may be the Initiating Device A. In a medical device system in which the IMD 210 is a battery powered device, it may be desired to have the IMD 210 function as the Non-Initiating Device B to minimize the battery drain of the IMD 210. Alternatively, it may be desired to have the IMD 210 drive the authentication process based on the security platform implemented.

In FIG. 4A at operation 402, the IMD 210 and the ED 250 exchange pairing features. If the numeric comparison authentication method is a numeric comparison Bluetooth native authentication method, both the IMD 210 and the ED 250 establish their Bluetooth® Low Energy (BLE) IO capabilities or IOCaps on DisplayYesNo or KeyboardDisplay even though one or both of the IMD 210 and the ED 250 do not have either of the capabilities. The establishing of the IOCaps by the devices forces the devices to perform authentication using numeric comparison. The mutual authentication then proceeds to secure pairing. As part of secure pairing, both the IMD 210 and the ED 250 may generate random private keys using cryptographically secure random number generators (CSRNGs) or cryptographically secure pseudo-random number generators (CSPRNGs). Each device then generates matching public keys. In some examples the public keys are calculated using an elliptic curve Diffie-Hellman (ECDH) algorithm.

Returning to FIG. 3 at block 310, the IMD 210 and the ED 250 exchange public keys as part of the secure pairing. This is shown in FIG. 4A as an exchange of public keys PKa and PKb at operation 404. In some examples, the IMD 210 and the ED 250 perform operation 406 in which the IMD 210 and the ED 250 verify that the key received in the exchange belongs to the elliptic curve it used to generate its own public key. For instance, the processing circuitry of the IMD verifies that that ED public key it received from the ED 250 belongs to the corresponding elliptic curve the IMD 210 used to generate the IMD public key. This is shown in operation 406 as each of the devices computing a Diffie-Hellman algorithm public key (DHKey).

At operation 408, each of the IMD 201 and ED 250 generate a first nonce. A nonce is a multi-bit arbitrary binary number or random number that is used just once. In certain examples, the IMD 210 and ED 250 each generate a 128-bit nonce. The IMD 201 and ED 250 exchange the first nonces they generated. At operation 412, this is shown as an exchange of nonce Na and nonce Nb.

In certain examples, one of the IMD 210 or ED 250 may optionally compute a commitment value. At operation 410, this is shown as Non-Initiating Device B computing commitment value Cb. The commitment value is a one-way function (f1) of the exchanged public keys (PKa, PKb) and the first nonce Nx of the device computing the commitment value, or as shown in operation 410,

$$Cb = f 1 ( PKax , PKbx , Nb , 0 ) .$$

The computed commitment value is sent to the other device. In FIG. 4A, Initiating Device A is the device receiving the commitment value.

At operation 414, the device receiving the commitment value verifies the commitment value using the public keys and the first nonce received from the sending device. For example, the IMD 210 may receive an ED commitment value from the ED that includes a one-way function of the IMD public key, the ED public key, and the first ED nonce. The IMD 210 verifies the commitment value using its IMD public key, the ED public key received in the exchange of public keys at operation 404, and the ED nonce received in the nonce exchange at operation 412. The IMD 210 continues communication with the ED 250 when verifying the commitment value (e.g., Cb) received from the ED 250. and ends the communication with the ED 250 when not verifying the ED commitment value.

Returning to FIG. 3 at block 315, both the IMD 210 and ED 250 produce a confirmation value. The confirmation value is generated by each device using both the IMD public key and the ED public key. In certain examples, each device computes the confirmation value using the IMD public key, the first IMD nonce, the ED public key, and the first ED nonce. This is shown in FIG. 4A at operation 416. Initiating Device A computes a confirmation value (Va) that is a function (g) of the exchanged public keys (PKa, PKb) and the exchanged first nonces (Na, Nb) or

$$Va = g ( PKax , PKbx , Na , Nb ) .$$

The non-initiating device computes confirmation value (Vb) that is the same function of the exchanged public keys (PKa, PKb) and the exchanged first nonces (Na, Nb). In some examples, the confirmation values (Va, Vb) are 6-digit confirmation values.

Operation 418 of FIG. 4B shows that the confirmation values (Va, Vb) generated at this point are not exchanged between the IMD 210 and the ED 250. Unsigned confirmation values are not exchanged. Instead, both devices automatically accept that the confirmation values generated are the same. According to some examples, the secure pairing and public key exchange may be performed using functions within a BLE stack of the IMD and ED. The computation and verification of the commitment value in operations 410 to 414 may also be handled within the BLE stack. Operation 418 is not a conventional BLE function and is performed outside the BLE stacks as are the subsequent operations in FIG. 4B.

Returning to FIG. 3 at block 320, each of the IMD 210 and ED 250 signs its respective computed confirmation value using its own securely generated private signing key. In some examples, the computed confirmation value is combined with another numerical value before it is signed. In certain examples, this other numerical value is a second nonce received from the other device.

At operation 420 in FIG. 4B, each of the IMD 210 and ED 250 generates a second nonce and the devices exchange their second nonces. The second nonces may also be 128-bit nonces. Each device concatenates its generated confirmation value with the received second nonce and the result is signed using its private key. For example, IMD 210 may be the Initiating Device A in FIG. 4B that generates a second IMD nonce (Nc) and sends the second IMD nonce to the ED 250 in operation 420. The ED 250 may be the Non-Initiating Device B that generates a second ED nonce (Nd) and sends the second ED nonce to the IMD 210.

At operation 422, the IMD 210 concatenates its generated confirmation value with the received second ED nonce (Va, Nd) and the ED 250 concatenates its generated confirmation value with the received second IMD nonce (Vb, Nc). Each of the IMD 210 and ED 250 signs the concatenated result using its private signing key [Sa(Va, Nc)] or [Sb(Vb, Nd)] to produce a signed confirmation value (CVa, CVb).

Returning to FIG. 3 at block 325, the IMD 210 and ED 250 exchange signed confirmation values. If the signed confirmation values are verified at block 330, the inter-device communication continues at block 335. If a signed confirmation value is not verified at 330 by either device, that device ends its communication with the other device at block 340.

In FIG. 4B, the signed confirmation values (CVa, CVb) are exchanged at operation 424. At operation 426, each of the devices verifies the signed confirmation value received from the other side. For operation 418, it was noted that each of the devices accepts that its original unsigned confirmation value is the same as the confirmation value generated by the other device (or Va=Vb). Also, each of the devices has the second nonce included in the signed confirmation value because it sent the second nonce to the other device in the exchange at operation 420. The unsigned confirmation value and second nonce are used to verify the received signed confirmation value.

For example, in operation 426 the IMD 210 verifies the signed ED confirmation value when

$$( Va , Nc ) = S {a}^{- 1} ( C V b ) ,$$

and the ED 250 verifies the signed IMD confirmation value when

$$( Vb , Nc ) = S {b}^{- 1} ( C V a ) .$$

At operation 428, mutual device authentication is achieved when the verification is passed by both the IMD210 and the ED 250. The communication session continues when the mutual authentication passes. The communication session ends when verification is not passed by at least one of the devices. The final verification depends on multiple conditions: the message signature validates using the other device's public key, the signed confirmation value matches the device's own confirmation value, and the signed nonce matches the nonce originally generated. The communication session may be performed using the BLE communication protocol but not all of the operations of the mutual authentication are performed using a BLE stack. For example, the secure pairing and exchange of public keys operations may be functions within a BLE stack while the operations to produce, exchange, and verify signed confirmation values are performed outside a BLE stack.

The authentication techniques described achieve a robust automatic mutual device-identity authentication and protects against hostile attacks such as man-in-the-middle attacks. Primary cryptographic objectives of confidentiality, integrity, authenticity, and non-repudiation are achieved. The authentication techniques can be integrated with existing cybersecurity infrastructure. The solution integrates readily into systems (e.g., systems utilizing BLE cryptography), minimizing barriers to the authentication implementation barriers. Devices authenticate automatically without user intervention. This background authentication process eliminates manual steps while maintaining robust security, enhancing both usability and efficiency. The system operates through a single communication channel between the IMD and ED, reducing hardware complexity. This simplification translates to lower costs, smaller device footprints, and reduced system complexity.

### ADDITIONAL DESCRIPTION AND EXAMPLES

A first Example (Example 1) includes subject matter (such as an implantable medical device or IMD) comprising a radio frequency (RF) transceiver to communicate with an external device (or ED) and processing circuitry operatively coupled to the RF transceiver. The processing circuitry is configured to enter a numeric comparison authentication mode; send an IMD public key to the ED and receive an ED public key from the ED; produce an IMD confirmation value using both the IMD public key and the ED public key; sign the IMD confirmation value using an IMD private key; send a signed IMD confirmation value to the ED and receive a signed ED confirmation value from the ED; and continue communication with the ED when verifying the signed ED confirmation value and end the communication with the ED when not verifying the signed ED confirmation value.

In Example 2, the subject matter of Example 1 optionally includes a memory containing instructions, that when performed by the processing circuitry, cause the IMD to set an input-output (IO) capability of the IMD even though the IMD does not have the IO capability. The processing circuitry is optionally configured to enter the numeric comparison authentication mode when performing the instructions.

In Example 3, the subject matter of one or both of Examples 1 and 2 optionally includes processing circuitry configured to determine a first IMD nonce that is a multi-bit arbitrary binary number; send the first IMD nonce to the ED and receive a first ED nonce from the ED; produce the IMD confirmation value using the IMD public key, the ED public key, the first IMD nonce, and the first ED nonce.

In Example 4, the subject matter of Example 3 optionally includes processing circuitry configured to receive an ED commitment value from the ED that includes a one-way function of the IMD public key, the ED public key, and the first ED nonce; and continue communication with the ED when verifying the ED commitment value and ending the communication with the ED when not verifying the ED commitment value.

In Example 5, the subject matter of one or both of Examples 3 and 4 optionally includes processing circuitry configured to compute an IMD commitment value that is a one-way function of the IMD public key, the ED public key, and the first IMD nonce; and send the IMD commitment value to the ED.

In Example 6, the subject matter of one or any combination of Examples 3-5 optionally includes processing circuitry configured to send a second IMD nonce to the ED and receive a second ED nonce from the ED; concatenate the IMD confirmation value with the second ED nonce; and sign the concatenated IMD confirmation value and the second ED nonce using the IMD private key to produce the signed IMD confirmation value.

In Example 7, the subject matter of Example 6 optionally includes processing circuitry configured to verify the signed ED confirmation value by verifying a signature in the signed ED confirmation value it receives is valid using the ED public key, verifying an ED confirmation value of the signed ED confirmation value matches the IMD confirmation, and verifying the second nonce of the signed ED confirmation value matches the second IMD nonce sent to the IMD.

In Example 8, the subject matter of one or both of Examples 6 and 7 optionally includes processing circuitry configured to calculate the IMD public key using an elliptic curve Diffie-Hellman (ECDH) public key algorithm, and verify that the ED public key is an ECDH public key.

In Example 9, the subject matter of one or any combination of Examples 1-8 optionally includes a Bluetooth Low Energy (BLE) stack and an RF transceiver configured to communicate with the ED using a communication protocol.

In Example 10, the subject matter of Example 9 optionally includes performing the secure pairing of the IMD and the ED using functions within the BLE stack, and performing mutual verification by the processing circuitry outside the BLE stack using the signed IMD confirmation value and the signed ED confirmation value.

Example 11 includes subject matter (such as a method of mutual authentication between an IMD and an ED) or can optionally be combined with one or any combination of Examples 1-10 to include such subject matter, comprising putting the IMD and the ED into a numeric comparison authentication mode; performing secure pairing that includes the IMD and ED exchanging public keys; producing, by both the IMD and ED, a respective confirmation value using both the public keys, and both the IMD and ED signing its respective confirmation value using its own private signing key; exchanging signed confirmation values; and continuing communication between the IMD and ED when both the IMD and ED verify a received signed confirmation value and ending communication when at least one of the IMD and ED does not verify the received signed confirmation value.

In Example 12, the subject matter of Example 11 optionally includes causing both of the IMD and ED to establish input-output (IO) capabilities even though one or both of the IMD and ED do not have the IO capabilities.

In Example 13, the subject matter of one or both of Examples 11 and 12 optionally includes the IMD and ED exchanging first nonces, and the IMD and ED each generating the confirmation value using both public keys and both first nonces and not exchanging the confirmation value.

In Example 14, the subject matter of Example 13 optionally includes one of the IMD or ED computing a commitment value that is a one-way function of the public keys and the first nonce of the one of the IMD or ED, and sending the commitment value to a paired IMD or ED; and verifying, by the paired IMD or ED, the commitment value.

In Example 15, the subject matter of one or both of Examples 13 and 14 optionally includes the IMD and ED exchanging second nonces; and each of the IMD and ED concatenating the confirmation value it produced with a received second nonce and signing the concatenated confirmation value and received second nonce using its own private signing key.

In Example 16, the subject matter of Example 15 optionally includes each of the IMD and ED verifying a signature in a signed confirmation value it receives is valid using a received public key; each of the IMD and ED verifying a signed confirmation value it receives matches its respective confirmation value; and each of the IMD and ED verifying a signed second nonce it receives in the signed confirmation value matches the second nonce it generated.

In Example 17, the subject matter of one or any combination of Examples 11-16 optionally includes the IMD and ED each calculating a matching public key using an elliptic curve Diffie-Hellman (ECDH) public key algorithm, exchanging the ECDH public keys, and verifying a received public key is an ECDH public key.

In Example 18, the subject matter of one or any combination of Examples 11-17 optionally includes the IMD and ED exchanging the public keys and the signed confirmation values using a Bluetooth Low Energy (BLE) communication protocol.

In Example 19, the subject matter of one or any combination of Examples 11-18 optionally includes performing the secure pairing within a BLE stack of the IMD and ED, and producing a confirmation value and exchanging signed communication values outside the BLE stack of the IMD and ED.

Example 20 includes subject matter (such as an external device (ED) of a medical system) or can optionally be combined with one or any combination of Examples 1-19 to include such subject matter, comprising an RF transceiver to communicate with an implantable medical device (IMD) of the medical device system; processing circuitry operatively coupled to the RF transceiver; a memory containing instructions, that when performed by the processing circuitry, cause the processing circuitry to enter a numeric comparison authentication session with the IMD and perform operations including: sending an ED public key to the IMD and receive an IMD public key from the IMD; producing an ED confirmation value using both the ED public key and the IMD public key; signing the ED confirmation value using an ED private key; sending a signed ED confirmation value to the IMD and receiving a signed IMD confirmation value from the IMD; and continuing communication with the IMD when verifying the signed IMD confirmation value and ending the communication with the IMD when not verifying the signed IMD confirmation value.

In Example 21, the subject matter of Example 20 optionally includes the memory further includes instructions that cause the processing circuitry to perform operations including entering a numeric comparison authentication mode by establishing input-output capabilities of the ED.

In Example 22, the subject matter of one or both of Examples 20 and 21 optionally includes the memory includes instructions that cause the processing circuitry to perform operations including: determining a first ED nonce; sending the first ED nonce to the IMD and receiving a first IMD nonce from the IMD; producing the ED confirmation value using the ED public key, the IMD public key, the first ED nonce, and the first IMD nonce; sending a second ED nonce to the IMD and receive a second IMD nonce from the IMD; concatenating the ED confirmation value with the second IMD nonce; and signing the concatenated the ED confirmation value and the second IMD nonce using the ED private key to produce the signed ED confirmation value.

These non-limiting Examples can be combined in any permutation or combination. The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An implantable medical device (IMD) comprising:
a radio frequency (RF) transceiver to communicate with an external device (ED);
processing circuitry operatively coupled to the RF transceiver and configured to:
enter a numeric comparison authentication mode;
send an IMD public key to the ED and receive an ED public key from the ED;
produce an IMD confirmation value using both the IMD public key and the ED public key;
sign the IMD confirmation value using an IMD private key;
send a signed IMD confirmation value to the ED and receive a signed ED confirmation value from the ED; and
continue communication with the ED when verifying the signed ED confirmation value and end the communication with the ED when not verifying the signed ED confirmation value.

2. The IMD of claim 1, including:
a memory containing instructions, that when performed by the processing circuitry, cause the IMD to set an input-output (IO) capability of the IMD even though the IMD does not have the IO capability; and
wherein the processing circuitry is configured to enter the numeric comparison authentication mode when performing the instructions.

3. The IMD of claim 1, wherein the processing circuitry is configured to:
determine a first IMD nonce that is a multi-bit arbitrary binary number;
send the first IMD nonce to the ED and receive a first ED nonce from the ED; and
produce the IMD confirmation value using the IMD public key, the ED public key, the first IMD nonce, and the first ED nonce.

4. The IMD of claim 3, wherein the processing circuitry is configured to:
receive an ED commitment value from the ED that includes a one-way function of the IMD public key, the ED public key, and the first ED nonce; and
continue communication with the ED when verifying the ED commitment value and ending the communication with the ED when not verifying the ED commitment value.

5. The IMD of claim 3, wherein the processing circuitry is configured to:
compute an IMD commitment value that is a one-way function of the IMD public key, the ED public key, and the first IMD nonce; and
send the IMD commitment value to the ED.

6. The IMD of claim 3, wherein the processing circuitry is configured to:
send a second IMD nonce to the ED and receive a second ED nonce from the ED;
concatenate the IMD confirmation value with the second ED nonce; and
sign the concatenated IMD confirmation value and the second ED nonce using the IMD private key to produce the signed IMD confirmation value.

7. The IMD of claim 6, wherein the processing circuitry is configured to verify the signed ED confirmation value by:
verifying a signature in the signed ED confirmation value it receives is valid using the ED public key;
verifying an ED confirmation value of the signed ED confirmation value matches the IMD confirmation value; and
verifying the second nonce of the signed ED confirmation value matches the second IMD nonce sent to the IMD.

8. The IMD of claim 6, wherein the processing circuitry is configured to:
calculate the IMD public key using an elliptic curve Diffie-Hellman (ECDH) public key algorithm; and
verify that the ED public key is an ECDH public key.

9. The IMD of claim 1, including a Bluetooth Low Energy (BLE) stack; and
wherein the RF transceiver is configured to communicate with the ED using a communication protocol.

10. The IMD of claim 9,
wherein secure pairing of the IMD and the ED is performed using functions within the BLE stack, and
wherein mutual verification using the signed IMD confirmation value and the signed ED confirmation value is performed by the processing circuitry outside the BLE stack.

11. A method of mutual authentication between an implantable medical device (IMD) and an external device (ED), the method comprising:
putting the IMD and the ED into a numeric comparison authentication mode;
performing secure pairing that includes the IMD and ED exchanging public keys;
producing, by both the IMD and ED, a respective confirmation value using both the public keys, and both the IMD and ED signing its respective confirmation value using its own private signing key;
exchanging signed confirmation values; and
continuing communication between the IMD and ED when both the IMD and ED verify a received signed confirmation value and ending communication when at least one of the IMD and ED does not verify the received signed confirmation value.

12. The method of claim 11, wherein the putting the IMD and ED into the numeric comparison authentication mode includes causing both of the IMD and ED to establish input-output (IO) capabilities even though one or both of the IMD and ED do not have the IO capabilities.

13. The method of claim 11, wherein producing the confirmation value includes:
the IMD and ED exchanging first nonces; and
the IMD and ED each generating the confirmation value using both public keys and both first nonces and not exchanging the confirmation value.

14. The method of claim 13, including:
one of the IMD or ED computing a commitment value that is a one-way function of the public keys and the first nonce of the one of the IMD or ED, and sending the commitment value to a paired IMD or ED; and
verifying, by the paired IMD or ED, the commitment value.

15. The method of claim 13, wherein signing a confirmation value includes:
the IMD and ED exchanging second nonces; and
each of the IMD and ED concatenating the confirmation value it produced with a received second nonce and signing the concatenated confirmation value and received second nonce using its own private signing key.

16. The method of claim 15,
each of the IMD and ED verifying a signature in a signed confirmation value it receives is valid using a received public key;
each of the IMD and ED verifying a signed confirmation value it receives matches its respective confirmation value; and
each of the IMD and ED verifying a signed second nonce it receives in the signed confirmation value matches the second nonce it generated.

17. The method of claim 11, wherein the performing secure pairing includes the IMD and ED each calculating a matching public key using an elliptic curve Diffie-Hellman (ECDH) public key algorithm, exchanging the ECDH public keys, and verifying a received public key is an ECDH public key.

18. The method of claim 11, wherein the exchanging the public keys and the signed confirmation values includes the IMD and ED exchanging the public keys and the signed confirmation values using a Bluetooth Low Energy (BLE) communication protocol.

19. The method of claim 11, wherein the performing the secure pairing includes performing the secure pairing within a BLE stack of the IMD and ED and wherein the producing a confirmation value and exchanging signed communication values includes producing a confirmation value and exchanging signed communication values outside the BLE stack of the IMD and ED.

20. An external device (ED) of a medical device system, the ED comprising:
a radio frequency (RF) transceiver to communicate with an implantable medical device (IMD);
processing circuitry operatively coupled to the RF transceiver;
a memory containing instructions, that when performed by the processing circuitry, cause the processing circuitry to enter a numeric comparison authentication session with the IMD and perform operations including;
sending an ED public key to the IMD and receive an IMD public key from the IMD;
producing an ED confirmation value using both the ED public key and the IMD public key;
signing the ED confirmation value using an ED private key;
sending a signed ED confirmation value to the IMD and receiving a signed IMD confirmation value from the IMD; and
continuing communication with the IMD when verifying the signed IMD confirmation value and ending the communication with the IMD when not verifying the signed IMD confirmation value.

21. The ED of claim 20, wherein the memory further includes instructions that cause the processing circuitry to perform operations including:
entering a numeric comparison authentication mode by establishing input-output capabilities of the ED.

22. The ED of claim 20, wherein the memory further includes instructions that cause the processing circuitry to perform operations including:
determining a first ED nonce;
sending the first ED nonce to the IMD and receiving a first IMD nonce from the IMD;
producing the ED confirmation value using the ED public key, the IMD public key, the first ED nonce, and the first IMD nonce;
sending a second ED nonce to the IMD and receive a second IMD nonce from the IMD;
concatenating the ED confirmation value with the second IMD nonce; and
signing the concatenated the ED confirmation value and the second IMD nonce using the ED private key to produce the signed ED confirmation value.
